(19) United States
(12) Patent Application Publication
(10) Pub. No.: US 2026/0364381 A1
(43) Pub. Date: Nov. 19, 2026

(54) SYSTEM AND METHOD FOR X-RAY IMAGING AND TARGETED X-RAY THERAPY

(71) Applicant: NCX Corporation, Raleigh, NC (US)

(72) Inventors: Jian ZHANG, Durham, NC (US); Derrek SPRONK, Chapel Hill, NC (US); John HAND, Carthage, NC (US)

(21) Appl. No.: 19/486,715

(22) PCT Filed: Jul. 16, 2024
(86) PCT No.: PCT/IB2024/056901

§ 371 Date: Nov. 21, 2025

Related U.S. Application Data
(60) Provisional application No. 63/514,012, filed on Jul. 17, 2023.

(51) Int. Cl. A61N 5/10; H01J 35/06; H05G 1/58

(52) U.S. Cl. CPC A61N 5/107; A61N 5/1039; A61N 5/1042; A61N 5/1083; H01J 35/065; H05G 1/58; A61N 2005/1054

(57) ABSTRACT

An X-ray therapy method using an X-ray device includes arranging a cathode device of an X-ray source into an imaging mode to focus electrons on a focal area with a first size on an anode. The X-ray source in the imaging mode is actuated to emit an X-ray beam toward an X-ray detector to obtain a first X-ray image of a target. The X-ray source or object is adjusted to register the target with a diagnostic image thereof, and to direct the X-ray source toward the target, based on a comparison of the first X-ray image and diagnostic image. The cathode device is switched into a power mode to focus electrons on a second focal area, having a second size greater than the first size, on the anode. The X-ray source in the power mode is actuated to deliver a therapeutic X-ray dosage to the target.

### BACKGROUND

### Field of the Disclosure

Aspects of the present disclosure are directed to X-ray imaging and therapy and, more particularly, to a system and method for X-ray imaging and targeted X-ray therapy.

### Description of Related Art

Radiation therapy (RT) is generally considered an effective treatment option for controlling tumors locally. Many cancer patients incorporate RT as part of their overall cancer management plan. However, despite its ability to kill cancer cells, RT does not always yield successful outcomes. This is often due to the fact that the radiation dose required to eradicate the tumor could cause significant damage to the surrounding healthy tissue, both in the short term and in the long term.

In recent years, there have been notable technological advancements in radiotherapy, particularly in terms of precise tumor coverage and minimizing exposure to healthy tissue during treatment planning and delivery. As a result, cancer patients now have access to conformal and intensity-modulated radiotherapy (IMRT) as well as imaging-guided radiotherapy (IGRT).

While state-of-the-art radiotherapy offers promising benefits for many cancer patients, these benefits are often limited by the necessity of high radiation doses that could cause intolerable harm to critical structures near the tumor site. Additionally, current radiotherapy techniques may be less effective in pediatric patients, whose developing normal tissues are often more sensitive to radiation than their tumors. Therefore, pediatric patients are generally unable to tolerate radiotherapy doses that could be curative for adults with the same disease.

An alternative treatment approach called Spatially Fractionated Radiation Therapy (SFRT) has shown promise in improving the sparing of normal tissue and has yielded encouraging results. In conventional radiation therapy, a broad and continuous radiation beam is typically used for cancer treatment. In contrast, SFRT employs specialized radiation characterized by unique spatial, temporal, and radiation dose patterns. In SFRT, the radiation field is discrete and composed of beams ranging from tens of micrometers to a few millimeters or even centimeters in width. The spacing between adjacent beams is typically a few times larger (e.g., 2-10 times) than the width of the beams themselves.

Several SFRT techniques, such as GRID, FLASH, LATTICE radiation therapy (LRT), and microbeam radiation therapy (MRT), are currently being studied in preclinical research, with some early clinical experience available. The primary goals of these SFRT techniques are generally to achieve improved tumor control and reduce damage to healthy tissue. While there may be shared mechanisms, understanding of these mechanisms is currently limited. By stander effects, abscopal effects, vascular damage, angiogenic responses, and immune responses have all been proposed as potential factors. Despite its potential as a cost-effective cancer treatment, SFRT has not yet been widely adopted in clinical practice mainly due to the lack of effective radiation delivery systems.

Most of the SFRT treatments today are carried out using conventional radiotherapy linear accelerators (LINACs). The dose rate of a state-of-art radiotherapy LINAC machine is about 5 Grays/minute (Gy/min). Recently, it has been discovered that a certain form of SFRT treatment called Flash radiotherapy produces encouraging treatment outcomes. It, however, requires a relatively high radiation dose rate (e.g., over 40 Gy/sec) during the treatment delivery. This magnitude of dose rate cannot be achieved with existing radiotherapy LINACs. In addition, clinical LINACs generally produce Megavolt (MV) range X-rays, which is not suitable for SFRT treatment because of the poor dose profile caused by the large footprint of high energy MV radiation. Studies have shown that high kV range (e.g., a few hundred kilovolts: 160 kV-800 kV) X-ray radiation is preferred for SFRT treatment.

Meanwhile, SFRT has been demonstrated using proton therapy and synchrotron-based systems. Those advanced machines can deliver SFRT treatment with an acceptable dose profile at high dose rate. Such machines are, however, relatively expensive. The average cost of a proton therapy machine is over 50 million dollars, and the average cost of a synchrotron facility is over 100 million dollars. The high cost of such systems severely hinders their clinical usage.

In some instances, conventional X-ray sources have also been considered for SFRT treatment. As shown, for example, in FIG. 1, a conventional X-ray source includes an electron cathode and an X-ray anode. The anode carries high voltage, for example, up to a few hundred kVs. Electrons emitted by the cathode are accelerated by the anode voltage to high energy and to impact an area on the anode (focal spot) to generate X-ray radiation. Some of the X-ray sources may have a focusing electrode to adjust/control the size of the focal spot on the anode. Conventional X-ray sources are, however, relatively low in radiation dose rate. One reason for relatively low dose rate is that such conventional X-ray sources are generally designed for imaging applications which require relatively small focal spot sizes (e.g., sub-millimeter to a few millimeters in diameter). The small focal spots are preferred for providing high resolution imaging (e.g., a small focal spot size provides a higher pixel count proportional to the desired resolution). However, a small focal spot size also limits the maximum output power (dose rate) of the X-ray source, example, due to the thermal limit of the small focal spot. Many conventional X-ray sources are only capable of operating at relatively low current (e.g., mA range). Some high-power X-ray sources (e.g., with rotating anode) are designed to operate at a high peak power (e.g., up to 100 kW), but are generally limited to relatively short exposure time (e.g., 10's of milliseconds) and low duty cycle (e.g., a few percent).

An X-ray source with a relatively larger focal spot size or focal line (e.g., up to the 10's of centimeters) is required in order to deliver the power (dose rate) needed for SFRT treatment, particularly Flash radiotherapy. Some conventional X-ray sources have an electron beam focusing/de-focusing mechanism (electrostatic or magnetic) to adjust the focal spot size on the anode, as shown for example in FIG. 1. However, the range of focal spot size and configuration of the focal spot achievable by such a focusing/de-focusing mechanism is generally very limited (a factor of 2-3 in size range).

Further, modern radiotherapy requires imaging guidance in order to accurately deliver the radiation dose to the tumor. For SFRT treatment, it is even more critical to have imaging guidance during the treatment due to the small dimension of the radiation field (as small as tens of micrometers) and high dose rate (e.g., Flash radiotherapy requiring a dose rate of >40 Gy/sec). For some existing SFRT systems, the imaging guidance is generally achieved through additional standalone separate and discrete imaging devices, typically a separate X-ray imaging device combined with the radiotherapy device. The imaging device typically generates images from directions different from the treatment beams emitted by the radiotherapy device, very often orthogonal to the treatment beam, which is not ideal for imaging guidance and target tracking. Such an arrangement also complicates the overall system design and increases system costs.

Thus, there exists a need for an X-ray radiation treatment system that is adaptable to and capable of both radiation treatment and imaging functions, with minimal configuration changes between both operational modes. Such an X-ray radiation treatment system should include an X-ray source having a cathode device capable of operating in an imaging mode in which a small focal spot on the anode can be used to generate high resolution images, and capable of operating in a treatment mode in which a large focal spot/line on the anode can be used to achieve high radiation dosage rates desired for cancer treatment. Such an X-ray radiation treatment system should also include imaging guidance and feedback capabilities before and/or during the radiation treatment phases in order to ascertain that the radiation dose(s) are accurately delivered to the correct target location such that minimal healthy tissue is subject to the radiation treatment.

### BRIEF SUMMARY OF THE DISCLOSURE

The above and other needs are met by the present disclosure which, in one aspect, provides a method of providing multi-modal X-ray therapy to a target within an object using an X-ray device including an X-ray source and an X-ray detector, with the X-ray source including an anode and a field emission cathode device in spaced-apart relation with the anode and arranged to emit electrons toward the anode, and with the target being determined from and a diagnostic image of the target being obtained by diagnostic imaging of the object. Such a method comprises arranging the field emission cathode device of the X-ray source into an imaging mode to focus the electrons on a first focal area on the anode, the first focal area having a first focal area size; imaging the target by actuating the X-ray source in the imaging mode to emit an imaging X-ray beam toward the X-ray detector such that the imaging X-ray beam interacts with the target to obtain a first X-ray image of the target; adjusting the X-ray source or the object to register the target with the diagnostic image of the target, and to direct the X-ray source toward the target, in response to a comparison of the first X-ray image and the diagnostic image of the target; switching the arrangement of the field emission cathode device into a power mode to focus the electrons on a second focal area on the anode, the second focal area having a second focal area size greater than the first focal area size; and actuating the X-ray source in the power mode to emit a first therapeutic X-ray beam toward the target to deliver a first X-ray dosage to the target.

Another aspect of the present disclosure provides a multi-modal X-ray system for providing X-ray therapy to a target within an object, wherein the target is determined from and a diagnostic image of the target is obtained by diagnostic imaging of the object. Such an X-ray system comprises an X-ray detector; an X-ray source including an anode and a field emission cathode device in spaced-apart relation with the anode and arranged to emit electrons toward the anode; and a controller in communication with the X-ray source and the X-ray detector. The controller is configured to direct the X-ray source to arrange the field emission cathode device into an imaging mode to focus the electrons on a first focal area on the anode, the first focal area having a first focal area size; image the object by actuating the X-ray source in the imaging mode to emit an imaging X-ray beam such that the imaging X-ray beam interacts with the target and is detected by the X-ray detector to obtain a first X-ray image of the target; adjust the X-ray source or the object to register the target with the diagnostic image of the target, and direct the X-ray source toward the target, in response to a comparison of the first X-ray image and the diagnostic image of the target; direct the X-ray source to switch the arrangement of the field emission cathode device into a power mode to focus the electrons on a second focal area on the anode, the second focal area having a second focal area size greater than the first focal area size; and actuate the X-ray source in the power mode to emit a first therapeutic X-ray beam toward the target to deliver a first X-ray dosage to the target.

Still another aspect of the present disclosure provides a method of providing multiple modes of an X-ray source device, wherein the X-ray source device includes an anode and a field emission cathode device in spaced-apart relation with the anode and arranged to emit electrons toward the anode. Such a method comprises arranging the field emission cathode device into an imaging mode to focus the electrons on a first focal area on the anode, the first focal area having a first focal area size; and reversibly switching the arrangement of the field emission cathode device into a power mode to focus the electrons on a second focal area on the anode, wherein the second focal area has a second focal area size greater than the first focal area size.

Still another aspect of the present disclosure provides a multi-modal X-ray source device, comprising an anode; and a field emission cathode device in spaced-apart relation with the anode and arranged to emit electrons toward the anode, wherein the field emission cathode device is switchable between an imaging mode having the electrons focused on a first focal area on the anode, the first focal area having a first focal area size, and a power mode having the electrons focused on a second focal area on the anode, and wherein the second focal area has a second focal area size greater than the first focal area size.

The present disclosure thus includes, without limitation, the following example embodiments:

Example Embodiment 1: A method of providing multi-modal X-ray therapy to a target within an object using an X-ray device including an X-ray source and an X-ray detector, with the X-ray source including an anode and a field emission cathode device in spaced-apart relation with the anode and arranged to emit electrons toward the anode, and with the target being determined from and a diagnostic image of the target being obtained by diagnostic imaging of the object, the method comprising arranging the field emission cathode device of the X-ray source into an imaging mode to focus the electrons on a first focal area on the anode, the first focal area having a first focal area size; imaging the target by actuating the X-ray source in the imaging mode to emit an imaging X-ray beam toward the X-ray detector such that the imaging X-ray beam interacts with the target to obtain a first X-ray image of the target; adjusting the X-ray source or the object to register the target with the diagnostic image of the target, and to direct the X-ray source toward the target, in response to a comparison of the first X-ray image and the diagnostic image of the target; switching the arrangement of the field emission cathode device into a power mode to focus the electrons on a second focal area on the anode, the second focal area having a second focal area size greater than the first focal area size; and actuating the X-ray source in the power mode to emit a first therapeutic X-ray beam toward the target to deliver a first X-ray dosage to the target.

Example Embodiment 2: The method of any preceding example embodiment, or combinations thereof, comprising switching the arrangement of the field emission cathode device of the X-ray source into the imaging mode, after delivery of the first X-ray dosage to the target; re-imaging the target using the X-ray device to obtain a second X-ray image of the target; adjusting the X-ray source or the object to register the target with the diagnostic image of the target, and to direct the X-ray source toward the target, in response to a comparison of the second X-ray image and the diagnostic image of the target; switching the arrangement of the field emission cathode device into the power mode to focus the electrons on the second focal area on the anode; and actuating the X-ray source in the power mode to emit a second therapeutic X-ray beam toward the target to deliver a second X-ray dosage to the target.

Example Embodiment 3: The method of any preceding example embodiment, or combinations thereof, comprising arranging the X-ray detector to oppose the X-ray source when the field emission cathode device of the X-ray source is in a transmission imaging mode.

Example Embodiment 4: The method of any preceding example embodiment, or combinations thereof, comprising arranging the X-ray detector adjacent to the X-ray source when the field emission cathode device of the X-ray source is in a backscatter imaging mode.

Example Embodiment 5: The method of any preceding example embodiment, or combinations thereof, comprising arranging the X-ray detector to not receive the first or second therapeutic X-ray beam when the field emission cathode device of the X-ray source is in the power mode.

Example Embodiment 6: The method of any preceding example embodiment, or combinations thereof, wherein adjusting the X-ray source or the object comprises adjusting a distance between the X-ray source and the object, adjusting an angular position of the X-ray source in an orbit about the object, or adjusting a position of the X-ray source or the object relative to the other, laterally and non-parallel to the imaging X-ray beam.

Example Embodiment 7: The method of any preceding example embodiment, or combinations thereof, wherein a margin of the target is determined from the diagnostic imaging, and wherein the method comprises collimating the first or second therapeutic X-ray beam emitted by the X-ray source in the power mode such that a lateral dimension of the respective first and second therapeutic X-ray beam is not greater than a lateral dimension of the margin of the target perpendicular to the respective first and second therapeutic X-ray beam.

Example Embodiment 8: The method of any preceding example embodiment, or combinations thereof, wherein collimating the first or second therapeutic X-ray beam comprises collimating the first or second therapeutic X-ray beam emitted by the X-ray source in the power mode using a slit collimator such that the first or second therapeutic X-ray beam is emitted onto the target as a linear area.

Example Embodiment 9: The method of any preceding example embodiment, or combinations thereof, wherein actuating the X-ray source in the power mode to emit a first or second X-ray dosage to the target, comprises actuating the X-ray source in the power mode to emit a first or second X-ray dosage to the target at a predetermined X-ray dosage rate for a predetermined time to provide a cumulative X-ray dosage to the target.

Example Embodiment 10: The method of any preceding example embodiment, or combinations thereof, comprising performing imaging and re-imaging the target and delivering the first and second X-ray dosages using the same X-ray source.

Example Embodiment 11: The method of any preceding example embodiment, or combinations thereof, comprising collimating the first or second imaging X-ray beam emitted by the X-ray source in the imaging mode using a cone beam collimator such that the first or second imaging X-ray beam is emitted onto the object as an elliptical area or a circular area.

Example Embodiment 12: The method of any preceding example embodiment, or combinations thereof, comprising tracking movement of the object during delivery of the first X-ray dosage; and adjusting the X-ray source or the object to register the target with the diagnostic image of the target, and to maintain the X-ray source directed toward the target, in response to the tracked movement of the object.

Example Embodiment 13: A multi-modal X-ray system for providing X-ray therapy to a target within an object, the target being determined from and a diagnostic image of the target being obtained by diagnostic imaging of the object, the X-ray system comprising an X-ray detector; an X-ray source including an anode and a field emission cathode device in spaced-apart relation with the anode and arranged to emit electrons toward the anode; and a controller in communication with the X-ray source and the X-ray detector, the controller being configured to direct the X-ray source to arrange the field emission cathode device into an imaging mode to focus the electrons on a first focal area on the anode, the first focal area having a first focal area size; image the target by actuating the X-ray source in the imaging mode to emit an imaging X-ray beam such that the imaging X-ray beam interacts with the target and is detected by the X-ray detector to obtain a first X-ray image of the target; adjust the X-ray source or the object to register the target with the diagnostic image of the target, and direct the X-ray source toward the target, in response to a comparison of the first X-ray image and the diagnostic image of the target; direct the X-ray source to switch the arrangement of the field emission cathode device into a power mode to focus the electrons on a second focal area on the anode, the second focal area having a second focal area size greater than the first focal area size; and actuate the X-ray source in the power mode to emit a first therapeutic X-ray beam toward the target to deliver a first X-ray dosage to the target.

Example Embodiment 14: The system of any preceding example embodiment, or combinations thereof, wherein the controller is configured to direct the X-ray source to switch the arrangement of the field emission cathode device into the imaging mode, after the first X-ray dosage is delivered to the target; re-image the target using the X-ray device to obtain a second X-ray image of the target; adjust the X-ray source or the object to register the target with the diagnostic image of the target, and direct the X-ray source toward the target, in response to a comparison of the second X-ray image and the diagnostic image of the target;

direct the X-ray source to switch the arrangement of the field emission cathode device into a power mode to focus the electrons on the second focal area on the anode; and actuate the X-ray source in the power mode to emit a second therapeutic X-ray beam toward the target to deliver a second X-ray dosage to the target.

Example Embodiment 15: The system of any preceding example embodiment, or combinations thereof, wherein the X-ray detector is arranged to oppose the X-ray source when the field emission cathode device of the X-ray source is in a transmission imaging mode.

Example Embodiment 16: The system of any preceding example embodiment, or combinations thereof, wherein the X-ray detector is arranged adjacent to the X-ray source when the field emission cathode device of the X-ray source is in a backscatter imaging mode.

Example Embodiment 17: The system of any preceding example embodiment, or combinations thereof, wherein the X-ray detector is arranged to not receive the first or second therapeutic X-ray beam when the field emission cathode device of the X-ray source is in the power mode.

Example Embodiment 18: The system of any preceding example embodiment, or combinations thereof, wherein the controller is configured to adjust the X-ray source or the object by adjusting a distance between the X-ray source and the object, adjusting an angular position of the X-ray source in an orbit about the object, or adjusting a position of the X-ray source or the object relative to the other, laterally and non-parallel to the imaging X-ray beam.

Example Embodiment 19: The system of any preceding example embodiment, or combinations thereof, wherein a margin of the target is determined from the diagnostic imaging, and wherein the system comprises a collimator arranged between the X-ray source and the object, the collimator being configured to collimate the first or second therapeutic X-ray beam emitted by the X-ray source in the power mode such that a lateral dimension of the respective first and second therapeutic X-ray beam is not greater than a lateral dimension of the margin of the target perpendicular to the respective first and second therapeutic X-ray beam.

Example Embodiment 20: The system of any preceding example embodiment, or combinations thereof, wherein the collimator comprises a slit beam collimator configured to collimate the first or second therapeutic X-ray beam emitted by the X-ray source in the power mode such that the first or second therapeutic X-ray beam is emitted onto the target as a linear area.

Example Embodiment 21: The system of any preceding example embodiment, or combinations thereof, wherein the controller is configured to actuate the X-ray source in the power mode to emit a first or second X-ray dosage to the target at a predetermined X-ray dosage rate for a predetermined time to provide a cumulative X-ray dosage to the target.

Example Embodiment 22: The system of any preceding example embodiment, or combinations thereof, wherein imaging and re-imaging the target and delivering the first and second X-ray dosages is performed using the same X-ray source.

Example Embodiment 23: The system of any preceding example embodiment, or combinations thereof, comprising a cone beam collimator arranged between the X-ray source and the object, and configured to collimate the first or second imaging X-ray beam emitted by the X-ray source in the imaging mode such that the first or second imaging X-ray beam is emitted onto the object as an elliptical area or a circular area.

Example Embodiment 24: The system of any preceding example embodiment, or combinations thereof, comprising a tracking device in communication with the controller and configured to track movement of the object during delivery of the first X-ray dosage, wherein the controller is configured to adjust the X-ray source or the object to register the target with the diagnostic image of the target, and to maintain the X-ray source directed toward the target, in response to the tracked movement of the object.

Example Embodiment 25: A method of providing multiple modes of an X-ray source device, the X-ray source device including an anode and a field emission cathode device in spaced-apart relation with the anode and arranged to emit electrons toward the anode, comprising arranging the field emission cathode device into an imaging mode to focus the electrons on a first focal area on the anode, the first focal area having a first focal area size; and reversibly switching the arrangement of the field emission cathode device into a power mode to focus the electrons on a second focal area on the anode, the second focal area having a second focal area size greater than the first focal area size.

Example Embodiment 26: The method of any preceding example embodiment, or combinations thereof, wherein the field emission cathode device comprises a plurality of individually-controllable field emission cathodes, and wherein arranging the field emission cathode device into the imaging mode comprises actuating a first amount of the field emission cathodes to direct the electrons emitted thereby to the first focal area on the anode.

Example Embodiment 27: The method of any preceding example embodiment, or combinations thereof, wherein the field emission cathode device comprises a plurality of individually-controllable field emission cathodes, and wherein switching the arrangement of the field emission cathode device into the power mode comprises actuating a second amount of the field emission cathodes, the second amount being greater than the first amount, to direct the electrons emitted thereby to the second focal area.

Example Embodiment 28: The method of any preceding example embodiment, or combinations thereof, wherein directing the electrons to the second focal area comprises directing the electrons from each of the second amount of field emission cathodes to respective corresponding focal areas on the anode, the respective corresponding focal areas being adjacently disposed and arranged to form the second focal area on the anode.

Example Embodiment 29: The method of any preceding example embodiment, or combinations thereof, wherein each of the second amount of the field emission cathodes emits an electron current directed to the anode upon actuation thereof, and wherein the method comprises modulating the current emitted by a selected one or more of the second amount of field emission cathodes so as to modulate an intensity of the electrons emitted thereby to the second focal area.

Example Embodiment 30: A multi-modal X-ray source device, comprising: an anode; and a field emission cathode device in spaced-apart relation with the anode and arranged to emit electrons toward the anode, the field emission cathode device being switchable between an imaging mode having the electrons focused on a first focal area on the anode, the first focal area having a first focal area size, and a power mode having the electrons focused on a second focal area on the anode, the second focal area having a second focal area size greater than the first focal area size.

Example Embodiment 31: The device of any preceding example embodiment, or combinations thereof, wherein the field emission cathode device comprises a plurality of individually-controllable field emission cathodes configured such that the imaging mode comprises a first amount of the field emission cathodes actuated to direct the electrons emitted thereby to the first focal area on the anode.

Example Embodiment 32: The device of any preceding example embodiment, or combinations thereof, wherein the field emission cathode device is configured such that the power mode comprises a second amount of the field emission cathodes, the second amount being greater than the first amount, actuated to direct the electrons emitted thereby to the second focal area.

Example Embodiment 33: The device of any preceding example embodiment, or combinations thereof, wherein the second amount of field emission cathodes is arranged to direct the electrons emitted thereby to respective corresponding focal areas on the anode, the respective corresponding focal areas being adjacently disposed and arranged to form the second focal area on the anode.

Example Embodiment 34: The device of any preceding example embodiment, or combinations thereof, wherein each of the second amount of the field emission cathodes emits an electron current directed to the anode upon actuation thereof, and wherein the electron current emitted by a selected one or more of the second amount of field emission cathodes is modulatable so as to modulate an intensity of the electrons emitted thereby to the second focal area.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure, regardless of whether such features or elements are expressly combined or otherwise recited in a specific embodiment description herein. This disclosure is intended to be read holistically such that any separable features or elements of the disclosure, in any of its aspects and embodiments, should be viewed as intended, namely to be combinable, unless the context of the disclosure clearly dictates otherwise.

It will be appreciated that the summary herein is provided merely for purposes of summarizing some example aspects so as to provide a basic understanding of the disclosure. As such, it will be appreciated that the above described example aspects are merely examples and should not be construed to narrow the scope or spirit of the disclosure in any way. It will be appreciated that the scope of the disclosure encompasses many potential aspects, some of which will be further described below, in addition to those herein summarized. Further, other aspects and advantages of such aspects disclosed herein will become apparent from the following detailed description taken in conjunction with the accompanying drawings which illustrate, by way of example, the principles of the described aspects.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1 schematically illustrates a typical example prior art X-ray source including a metal anode (tungsten, molybdenum, etc.), an electron cathode, and in some of the examples, an electron-focusing electrode;

FIGS. 2A and 2B schematically illustrate an X-ray source according to one aspect of the present disclosure, wherein the field emission cathode device comprises a plurality of individually controllable field emission cathodes, wherein one of the field emission cathodes can be actuated to provide imaging X-rays from a first viewing angle (FIG. 2A), and then that field emission cathode deactuated and another one of the field emission cathodes actuated to provide imaging X-rays from a different viewing angle (FIG. 2B);

FIG. 3 schematically illustrates an X-ray source according to the aspect of the present disclosure shown in FIGS. 2A and 2B, wherein individual field emission cathodes (or small groups of field emission cathodes) can be sequentially actuated to provide imaging X-rays from multiple view viewing angles;

FIG. 4A schematically illustrates an X-ray source according to one aspect of the present disclosure, wherein the field emission cathode device comprises a plurality of individually controllable field emission cathodes, wherein many (or all) of the field emission cathode are simultaneously actuated to produce a large focal spot/line on the anode for delivery of radiotherapy treatment;

FIGS. 4B and 4C schematically illustrate an X-ray source according to the aspect of the present disclosure shown in FIG. 4A, wherein only some of the field emission cathodes (or some groups of field emission cathodes) are selectively actuated (with the remainder deactuated) in order to distribute the radiation dose delivery to the target (FIG. 4B), with the electron current from each field emission cathode or each group of field emission cathodes is modulated to produce intensity modulated radiation (IMRT) along the large focal spot/line;

FIG. 5 schematically illustrates a method of providing multiple operational modes of an X-ray source device, according to one aspect of the present disclosure;

FIG. 6A schematically illustrates an X-ray source arranged in a treatment/power mode, according to one aspect of the present disclosure, and actuated to deliver a radiotherapy treatment to the target;

FIGS. 6B and 6C schematically illustrate an X-ray source arranged in a treatment/power mode, according to the aspect of the present disclosure shown in FIG. 6A, wherein the X-ray source is configured to implement a single large focal spot/line on the anode and to emit X-ray radiation directed through a single slit collimator for delivering the radiotherapy treatment to the target (FIG. 6B), and wherein the X-ray source is configured to implement a multiple large focal spots/lines on the anode and to emit X-ray radiation directed through a slit collimator having multiple slits for delivering the radiotherapy treatment to the target (FIG. 6C);

FIGS. 7A and 7B schematically illustrate an X-ray source arranged in an imaging mode, according to one aspect of the present disclosure, wherein the X-ray source is configured to implement a small focal spot on the anode and to emit X-ray radiation directed through a cone beam collimator for imaging the target as shown from an axial view (FIG. 7A) and from a sagittal view (FIG. 7B);

FIG. 8A schematically illustrates a radiation treatment system including a multi-modal X-ray source, according to one aspect of the present disclosure;

FIGS. 8B and 8C schematically illustrate a radiation treatment system, according to the aspect of the present disclosure shown in FIG. 8A, wherein the X-ray source and X-ray detector are mounted to a gantry, wherein the gantry and/or the patient are movable relative to each other (FIG. 8B), or wherein the X-ray source and/or the X-ray detector are movable relative to each other (FIG. 8C), to facilitate alignment/positioning of the target relative to the X-ray source;

FIG. 9 schematically illustrates a method of providing multi-modal X-ray therapy to a target, according to one aspect of the present disclosure;

FIG. 10 schematically illustrates a radiation treatment system, according to the aspect of the disclosure shown in FIG. 8A, wherein multiple X-ray sources and corresponding multiple X-ray detectors opposing the X-ray detectors are mounted on the gantry;

FIG. 11 schematically illustrates a radiation treatment system, according one aspect of the disclosure, wherein an X-ray source is configured to form both imaging and treatment focal spots on the anode, and wherein a single collimator having both a cone beam collimator and a slit collimator is associated with the X-ray source such that imaging and radiation therapy treatment can be performed simultaneously;

FIG. 12(a)-12(c) schematically illustrate a radiation treatment system, according one aspect of the disclosure, wherein a single slit collimator is implemented for both treatment and imaging purposes;

FIGS. 13A and 13B schematically illustrate an image of the target/tumor generated by a radiation treatment system, according to the aspect of the disclosure shown in FIG. 12(a)-12(c), using a single slit collimator (FIG. 13A) for registration with the original diagnostic image of the target/tumor (FIG. 13B); and

FIG. 14 schematically illustrates a radiation treatment system, according one aspect of the disclosure, wherein an X-ray source is mounted on a robotic arm.

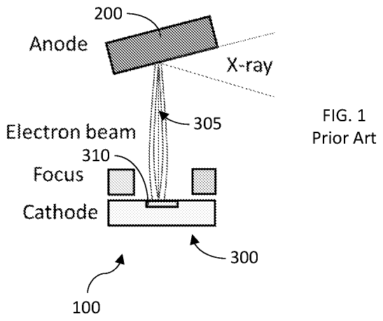

### DETAILED DESCRIPTION OF THE DISCLOSURE

The present disclosure now will be described more fully hereinafter with reference to the accompanying drawings, in which some, but not all embodiments of the disclosures are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

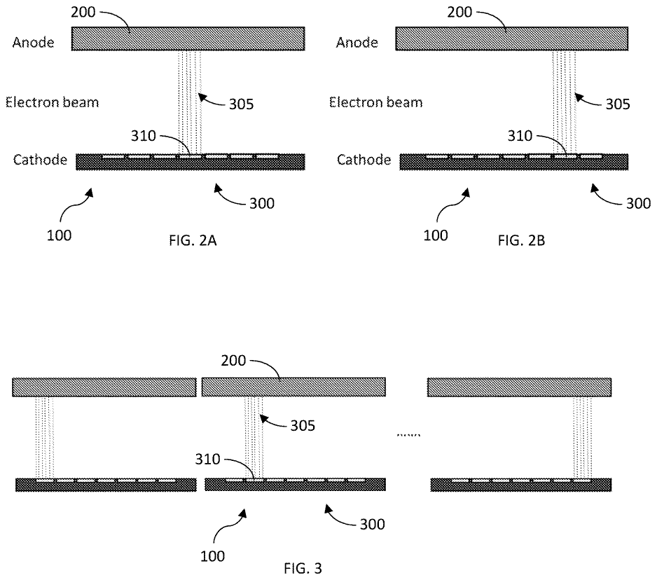

One aspect of the present disclosure comprises a multi-modal X-ray source device, generally indicated by the numeral 100(see, e.g., FIGS. 2A, 2B). In some aspects, such an X-ray source device 100 comprises an anode 200, and a field emission cathode device 300 in spaced-apart relation with the anode 200 and with the field emission cathode device 300 being arranged to emit electrons (e.g., an electron beam 305) toward the anode 200.

In one aspect, the field emission cathode device 300 comprises a plurality of individually-controllable field emission cathodes 310. As such, since X-ray imaging generally requires a small focal spot size on the anode 200 to provide higher resolution for the imaging, the X-ray source device 100 can be operated in an imaging mode, wherein only one field emission cathode 310 or a few, several, or otherwise a small number of field emission cathodes 310 of the field emission cathode device 300 can be actuated to achieve a relatively small focal spot (e.g., sub-millimeter to a few millimeters in diameter) of the electron beam(s) 305 on the anode 200 for imaging purposes (see, e.g., FIG. 2A) to obtain a view of the target from one imaging angle. In some configurations of the X-ray source 100, a different single field emission cathode 310 (or a different few, several, or otherwise a small number of field emission cathodes 310) may be actuated to achieve another view of the target from a different viewing angle, as shown in FIG. 2B. In the imaging mode, the energy of the X-ray source (e.g., the energy is proportional to the voltage applied to the anode 200) can be adjusted to an appropriate range for imaging (e.g., about 30 kV-160 kV) to achieve suitable X-ray imaging results. In some aspects, as shown for example in FIG. 3, multiple views of the target can be obtained from various viewing angles, for example, for three-dimensional (3D) imaging purposes. That is, as shown in FIG. 3, groups of one or a few field emission cathodes can be sequentially actuated to produce a series of X-ray images from different viewing angles. Those images can be processed and combined, as appropriate, to reconstructed and achieve a 3D view of the target object.

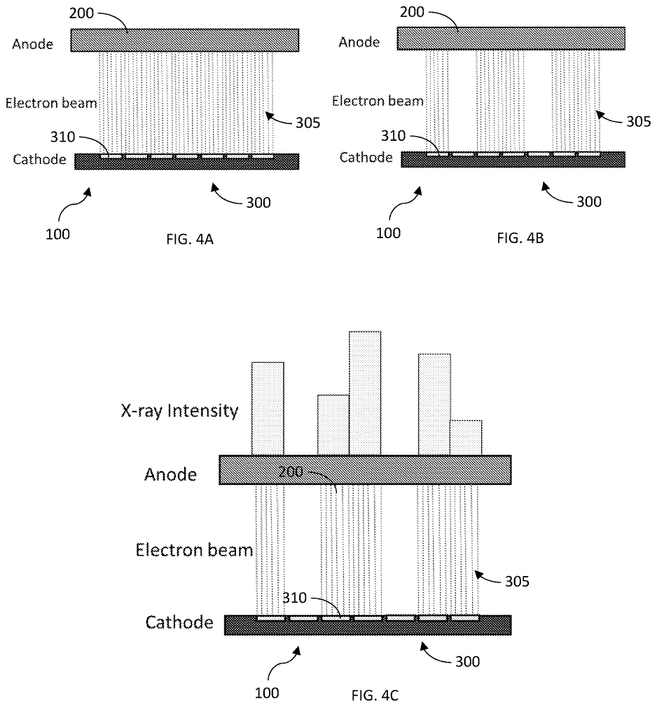

In another aspect of the present disclosure, the X-ray source 100 can be operated in a treatment mode or power mode, in order to produce a relatively large focal spot of the electron beam 305 on the anode 200. That is, in some instances, a large number (or all) of the plurality of field emission cathodes 310 can be actuated simultaneously, with the respective emitted electron beams being directed toward adjacent or overlapping focal spots on the anode 200, such that spots/areas of the anode 200 impacted by the electron beams 305 combine to form a joint large focal spot or focal line (e.g., up to the 10's of centimeters) on the anode 200, as shown for example in FIG. 4A. The large focal spot/line allows the X-ray source 100 to be operated at relatively higher power to provide a proportionally higher radiation dose rate (e.g., over 40 Gy/sec) as an output. For example, the energy of the X-ray source 100 is increased by increasing the voltage applied to the anode 200 to a relatively higher level (e.g., about 160 k V-800 kV) to achieve a suitable radiation dose rate distribution across the target for better treatment outcomes.

In some aspects, the X-ray source 100 in the treatment/power mode may have only some of the field emission cathodes 310 selectively actuated, while the remainder of the field emission cathodes 310 remain deactuated, as shown for example in FIG. 4B. In addition, the current (electron flow in the electron beam) from each of the actuated field emission cathodes (and thus the X-ray radiation emitted from the corresponding focal spot on the anode 200) can be programmed/modulated to have different intensity levels, as shown for example in FIG. 4C. This selective field emission cathode actuation and electron beam modulation operational mode of the X-ray source 100 results in different radiation intensities from the corresponding focal spots, as shown in FIG. 4C, and thereby allows an Intensity Modulated Radiation Therapy (IMRT) capability to be achieved in an SFRT treatment protocol.

Aspects of a multi-model X-ray source 100 may involve, for example, the field emission cathode device 300 being switchable between an imaging mode having the emitted electrons/electron beam 305 focused on a first focal area on the anode 200, wherein the first focal area has a first focal area size, and a power mode or treatment mode having the emitted electrons/electron beam 305 focused on a second focal area on the anode 200, wherein the second focal area has a second focal area size greater than the first focal area size.

In one aspect, the field emission cathode device 300 comprises a plurality of individually-controllable field emission cathodes 310 configured and arranged such that the imaging mode comprises a first amount of the field emission cathodes 310 that are actuated to direct the electrons emitted thereby (electron beam 305) to the first focal area on the anode 200. In a further aspect, the field emission cathode device 300 is configured such that the power mode comprises a second amount of the field emission cathodes 310 that are actuated to direct the electrons emitted thereby (electron beam 310) to the second focal area on the anode 200. The second amount of the field emission cathodes 310 is greater than the first amount of the field emission cathodes 310. Further, the second amount of field emission cathodes 310 is arranged to direct the electrons emitted thereby (electron beam or current) to respective corresponding focal areas on the anode 200, wherein the respective corresponding focal areas are adjacently disposed on the anode 200, and are arranged to form the second focal area on the anode 200. In further aspects, each cathode of the second amount of the field emission cathodes 310 is actuated to provide a current of electrons/an electron beam directed to the anode 200, and wherein the current from a selected one or more of the second amount of field emission cathodes 310 is modulatable so as to modulate an intensity of the electrons emitted thereby to the second focal area. As such, the first and second amounts of the field emission cathodes 305 in the field emission cathode device 300 can be selected and arranged to provide the appropriate focal spot size on the anode 200 for the imaging and treatment/power modes, respectively, with the appropriate energy for each mode being determined based on the voltage applied to the anode 200.

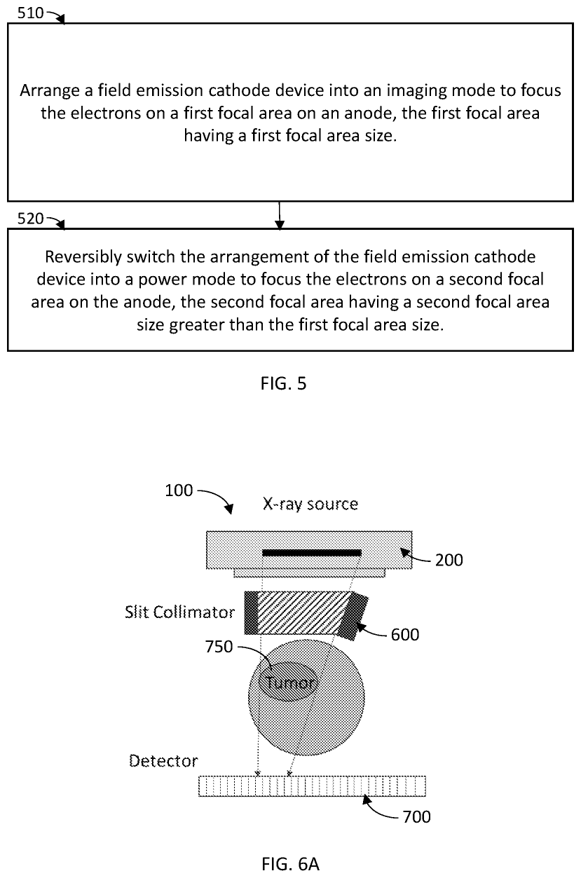

Another aspect of the present disclosure, as shown in FIG. 5, provides a method of providing multiple modes of an X-ray source device 100, wherein the X-ray source device 100 includes an anode 200 and a field emission cathode device 300 in spaced-apart relation with the anode 200 and arranged to emit electrons toward the anode 200. Such a method comprises arranging the field emission cathode device 300 into an imaging mode to focus the electrons on a first focal area on the anode 200, wherein the first focal area has a first focal area size (block 510); and reversibly switching the arrangement of the field emission cathode device 300 into a power mode to focus the electrons on a second focal area on the anode 200, wherein the second focal area has a second focal area size greater than the first focal area size (block 520).

In some aspects, the field emission cathode device 300 comprises a plurality of individually-controllable field emission cathodes 310, and the step of arranging the field emission cathode device 300 into the imaging mode comprises actuating a first amount of the field emission cathodes 310 to direct the electrons emitted thereby to the first focal area on the anode 200. In other aspects, the field emission cathode device 300 comprises a plurality of individually-controllable field emission cathodes 310, and the step of switching the arrangement of the field emission cathode device 300 into the power mode comprises actuating a second amount of the field emission cathodes 310, wherein the second amount is greater than the first amount, to direct the electrons emitted thereby to the second focal area. In still other aspects, the step of directing the electrons to the second focal area comprises directing the electrons from each of the second amount of field emission cathodes 310 to respective corresponding focal areas on the anode 200, wherein the respective corresponding focal areas are adjacently disposed on the anode 200 and arranged to or in combination form the second focal area on the anode. In further aspects, each of the second amount of the field emission cathodes 310 emits an electron current directed to the anode 310 upon actuation thereof, and the method comprises modulating the electron current emitted by a selected one or more of the second amount of field emission cathodes 310 so as to modulate an intensity of the electrons or electron current emitted thereby to the second focal area on the anode 200.

Aspects of the multi-modal X-ray source 100 disclosed herein thus include a capability of reversibly switching the same X-ray source between an imaging mode and a treatment/power mode by programming control of the field emission cathode device 300 to allow for selective actuation of one of more of the plurality of field emission cathodes 310 of the field emission cathode device 300. This selective actuation capability for the field emission cathodes 310 of the field emission cathode device 300 can be accomplished in many different manners such as disclosed, for example, in co-pending U.S. patent application Ser. No. 18/247,265, entitled “Multi-Beam X-ray Source and Method for Forming Same” and assigned to NCX Corporation, which is incorporated herein by reference. For example, multiple individually addressable and actuatable field emission cathodes can be implemented for multi-mode operation of the X-ray source 100. In this manner, the dual modes may be configured such that the size of the electron beam focal spot on the anode 200 can be varied as suitable such that a small focal spot is used for the imaging mode and a large focal spot is used for the radiotherapy treatment in the treatment/power mode.

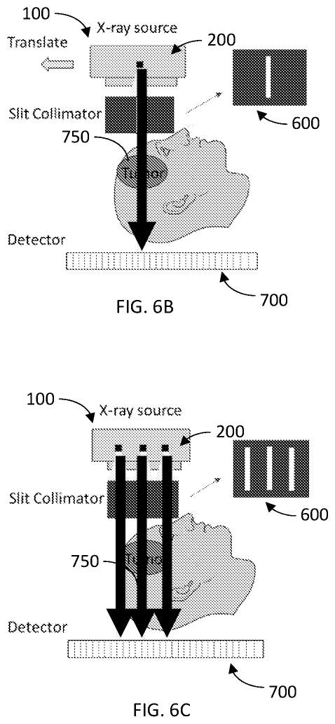

As shown for example in FIG. 6A, an X-ray source 100 configured to implement a large focal spot on the anode thereof is used for radiotherapy treatment with the X-ray source in the treatment/power mode. In some aspects, the large focal spot size may be cumulative in that the electron beam(s) can be directed to a single focal spot/line on the anode, or the electron beam(s) can be directed to multiple focal spots/lines on the anode, wherein the multiple focal spots/lines cumulatively provide the large focal spot of the X-ray source 100 in the treatment/power mode. In the treatment mode, a relatively higher radiation dose rate is often preferred for patient treatment (e.g., a tumor as the “target”). A large focal spot/line implemented via the X-ray source in the treatment mode allows the X-ray source 100 to operate at relatively higher power (higher anode voltage, higher electron current, and longer exposure time/duty cycle). A slit collimator 600 is utilized to generate the spatially discrete X-ray beams needed for SFRT treatment (e.g., for extending the radiation dose to the margins of the tumor/target). Though FIGS. 6A-6C illustrate a detector 700 opposing the X-ray source 100 such that the target/tumor 750 is disposed therebetween, in practice with the X-ray source 100 in treatment/power mode, the detector 700 is optional and can be relocated or removed. In addition to the X-ray source 100 in the treatment/power mode being arranged and configured such that the electron current is direct to a single focal spot/line or multiple focal spots/lines on the anode, the slit collimator 600 for directing the X-ray beam(s) emitted by the X-ray source 100 may be configured to have a single slit opening (see, e.g., FIG. 6B) or multiple slit openings (see, e.g., FIG. 6C). Should the emitted X-ray beam(s) directed through the slit collimator 600 not sufficiently extend to irradiate the tumor 750, the X-ray source 100 and/or the patient may be translated/moved (see, e.g., FIG. 6B) during the treatment procedure such that the X-ray beam(s) irradiate the entire tumor. Otherwise, the radiation therapy treatment can be divided into several sub-treatments, wherein each sub-treatment covers a portion of the target/tumor 750. That is, in such instances, the X-ray source 100 can be positioned and actuated to deliver a first radiation dose to a first portion of the target/tumor 750. The deactuated X-ray source 100 is then shifted/translated and then positioned and actuated to deliver a second radiation dose to a second portion of the target/tumor 750, with the process being repeated to provide radiation therapy treatment to the entire target/tumor 750.

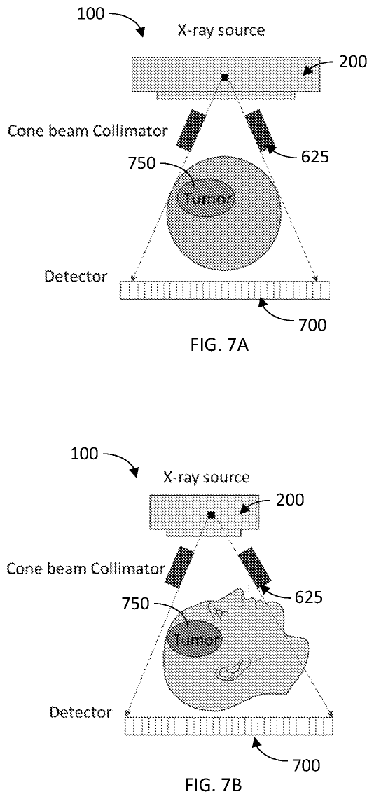

As shown for example in FIGS. 7A and 7B, an X-ray source 100 configured to implement a small focal spot on the anode thereof is used for imaging of the target/tumor 750 with the X-ray source in the imaging mode. The smaller focal spot on the anode is better suited for imaging purposes because it provides higher spatial resolution for the imaging. In addition, in the imaging mode, the X-ray source 100 may be operated at a relatively lower power (e.g., lower anode voltage, lower electron current, and shorter exposure time/duty cycle) so that the anode is able to withstand the power requirement associated with the smaller focal spot. In some aspects, a cone beam collimator 625 is used for directing the X-ray beam(s) emitted by the X-ray source 100 to define the field size for the imaging, as detected by the detector 700.

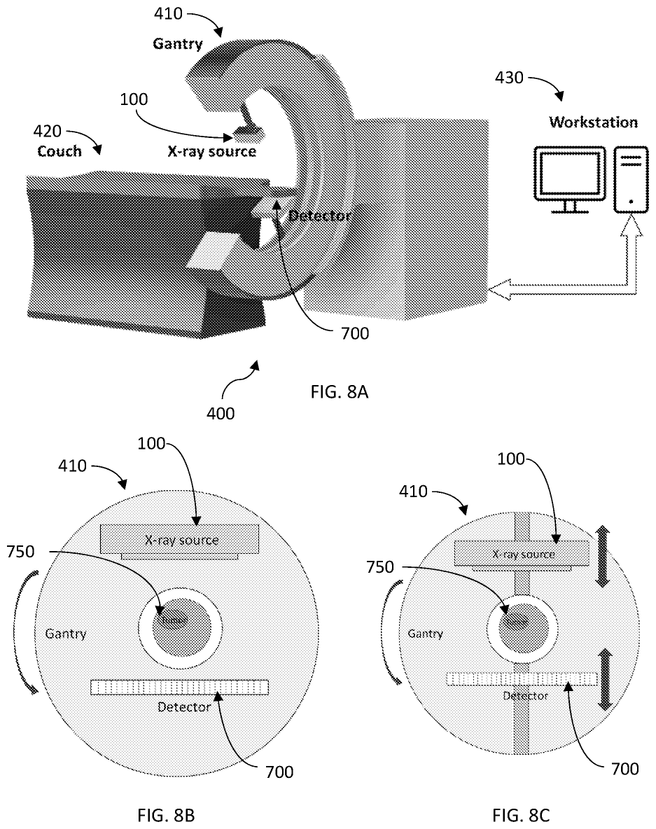

In some aspects, the multi-modal X-ray source 100 can be incorporated into a patient treatment system 400 as shown, for example, in FIG. 8A. Such a system 400 can include a gantry 410 for supporting the X-ray source 100 in opposing relation to the X-ray detector 700. A couch/table 420 is disposed adjacent to the gantry 410 for supporting the patient such that the target/tumor 750 is disposed between the X-ray source 100 and the X-ray detector 700. The couch/table 420 may be laterally movable (e.g., movable in a horizontal plane) to translate the patient into alignment with the treatment and/or imaging X-ray beam(s) emitted by the X-ray source 100. As shown in FIG. 8B, the gantry 410 can be configured to be rotatable about a medial axis extending therethrough, parallel to the couch/table 420, such that the X-ray source 100/X-ray detector 700 pair can be orbited about the patient to treat and/or image the tumor/target from different directions. In some instances, as shown in FIG. 8C, the gantry 410 may be configured to allow the X-ray source 100 and/or the X-ray detector 700 to be moved perpendicularly to the medial axis (e.g., shift perpendicularly or laterally to the medial axis). That is, FIG. 8C schematically illustrates that the X-ray source 100 and/or the X-ray detector 700 can be mounted rails 710 so that the respective distance thereof to the medial axis can be adjusted. The adjustable distances provide flexibility to achieve optimal treatment and/or imaging arrangements. For example, a relatively larger distance between the X-ray source 100 and the medial axis (e.g., the imaging center) will provide a larger radiation field (e.g., a dimensionally larger X-ray beam) for treatment and imaging. In another example, a relatively smaller distance between the X-ray source 100 and the medial axis/imaging center will increase the radiation dose rate for treatment and imaging.

A computer workstation/controller 430 is in communication with the couch/table 420, the gantry 410, the X-ray source 100 and X-ray detector 700 so as to manage, for example, radiation treatment planning, as well as radiation treatment delivery and control. Such functionality includes, for example, aligning and moving the patient via the couch/table 420 and/or the gantry 410, switching the X-ray source 100 between imaging mode and treatment/power mode, moving the X-ray detector 700 independently of the X-ray source 100 (e.g., to move the X-ray detector 700 away when the X-ray source 100 is in treatment/power mode), moving the X-ray detector 700 independently of or in conjunction with moving the X-ray source 100 (e.g., shifting the X-ray source 100 and/or the X-ray detector 700 perpendicularly or laterally to the medial axis), controlling the X-ray source 100 and the X-ray detector 700 as necessary in both the imaging mode and the treatment/power mode, and processing X-ray beams detected by the X-ray detector 700 form the desired imaging of the target 750. In particular aspects, the X-ray source 100 is controllable by the controller 430 such that the X-ray source 100, and thus the system 400, can be seamlessly switched between treatment/power mode and imaging mode to provide real time imaging guided radiation therapy (IGRT). In some aspects, a person of ordinary skill will appreciate that the switching between treatment/power mode and imaging mode in regard to the system 400 also includes the switching of collimators, for example, between a cone beam collimator (imaging) and a slit collimator (treatment/power).

In one example of a workflow associated with the treatment system 400 shown in FIG. 8A, an SFRT treatment plan is first generated using the workstation/controller 430 based on necessary system 400 and patient information. Once the treatment plan is completed, it is stored in the workstation/controller 430 and/or transferred to the system 400 for treatment delivery and control. Before starting the SFRT treatment, the patient is established on the couch/table 420 according to the treatment plan and configuration of the system 400. A set of X-ray images is then acquired with the X-ray source 100 operating in the imaging mode (small focal spot of the electron beam on the anode) to facilitate the patient setup (e.g., to determine the spatial location and breadth of the target/tumor with respect to the X-ray source 100). Once the patient is properly placed and aligned, the X-ray source 100 is switched to the treatment/power mode (large focal spot/line of the electron beam(s)on the anode) for the radiation therapy (SFRT) treatment. During the SFRT treatment, the operation of the system 400 with the X-ray source 100 in treatment/power mode can be paused, and the X-ray source 10 switched to imaging mode to confirm patient placement (e.g., the target being treated, a fiducial marker, etc.). If the patient/target placement does not correspond to the placement on which the treatment plan was formed, the patient/target is re-aligned and placed before the X-ray source 100 is switched back to the treatment/power mode to continue the SFRT treatment.

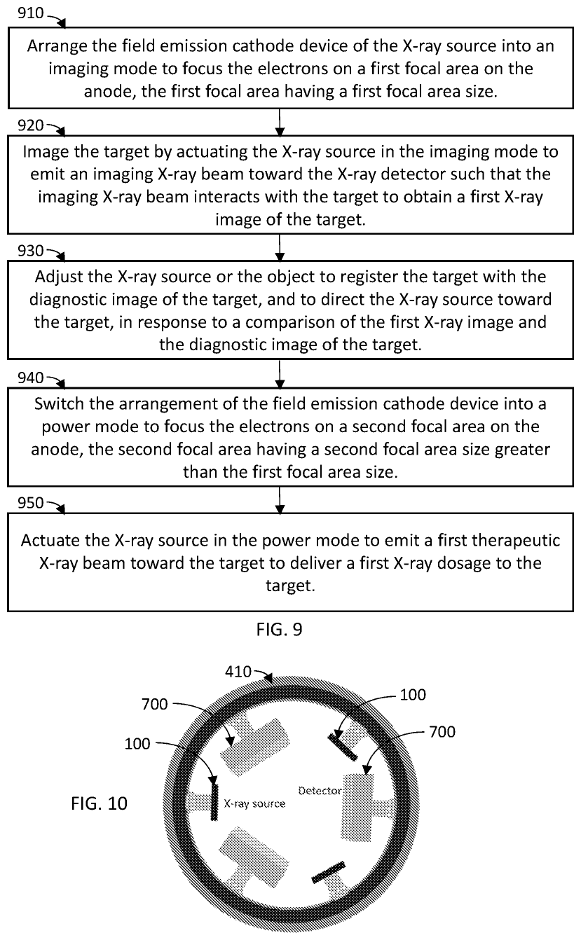

One aspect of the present disclosure, schematically illustrated in FIG. 9, comprises a method of providing multi-modal X-ray therapy to a target 750 within an object (e.g., patient) using an X-ray device including an X-ray source 100 and an X-ray detector 700, with the X-ray source 100 including an anode 200 and a field emission cathode device 310 in spaced-apart relation with the anode 200 and arranged to emit electrons 305 toward the anode 200, and with the target 750 being determined from and a diagnostic image of the target 750 being obtained by diagnostic imaging of the object. Such a method comprises arranging the field emission cathode device 310 of the X-ray source 100 into an imaging mode to focus the electrons 305 on a first focal area on the anode 200, the first focal area having a first focal area size (block 910); arranging the field emission cathode device 300 of the X-ray source 100 into an imaging mode to focus the electrons 305 on a first focal area on the anode 200, the first focal area having a first focal area size (block 920); imaging the target 750 by actuating the X-ray source 100 in the imaging mode to emit an imaging X-ray beam toward the X-ray detector 700 such that the imaging X-ray beam interacts with the target 750 to obtain a first X-ray image of the target 750 (block 930); adjusting the X-ray source 100 or the object to register the target 750 with the diagnostic image of the target 750, and to direct the X-ray source 100 toward the target 750, in response to a comparison of the first X-ray image and the diagnostic image of the target 750 (block 940); switching the arrangement of the field emission cathode device 300 into a power mode to focus the electrons 305 on a second focal area on the anode 200, the second focal area having a second focal area size greater than the first focal area size (block 950); and actuating the X-ray source 100 in the power mode to emit a first therapeutic X-ray beam toward the target 750 to deliver a first X-ray dosage to the target 750.

In some aspects, such a method comprises switching the arrangement of the field emission cathode device 300 of the X-ray source 100 into the imaging mode, after delivery of the first X-ray dosage to the target 750; re-imaging the target 750 using the X-ray device to obtain a second X-ray image of the target 750; adjusting the X-ray source 100 or the object to register the target 750 with the diagnostic image of the target 750, and to direct the X-ray source 100 toward the target 750, in response to a comparison of the second X-ray image and the diagnostic image of the target 750; switching the arrangement of the field emission cathode device 300 into the power mode to focus the electrons 305 on the second focal area on the anode 200; and actuating the X-ray source 100 in the power mode to emit a second therapeutic X-ray beam toward the target 750 to deliver a second X-ray dosage to the target 750.

In some aspects, the method comprises arranging the X-ray detector 700 to oppose the X-ray source 100 when the field emission cathode device 300 of the X-ray source 100 is in a transmission imaging mode. In other aspects, the method comprises arranging the X-ray detector 700 adjacent to the X-ray source 100 when the field emission cathode device 300 of the X-ray source 100 is in a backscatter imaging mode. In such instances, a backscatter element (not shown) may be provided and placed to oppose the X-ray source 100 such that the object/target is disposed therebetween, with the backscatter element being configured/arranged to reflect the imaging X-ray(s) back toward the X-ray detector 700 disposed adjacent to the X-ray source 100.

In some aspects, the method comprises arranging the X-ray detector 700 to not receive the first or second therapeutic X-ray beam when the field emission cathode device 300 of the X-ray source 100 is in the power mode. In other aspects, adjusting the X-ray source 100 or the object comprises adjusting a distance between the X-ray source 100 and the object, adjusting an angular position of the X-ray source 100 in an orbit about the object, and/or adjusting a position of the X-ray source 100 or the object relative to the other, laterally and non-parallel to the imaging X-ray beam.

In some aspects, a margin (e.g., boundary) of the target 750 is determined from the diagnostic imaging. In such aspects, the method comprises collimating the first or second therapeutic X-ray beam emitted by the X-ray source 100 in the power mode such that a lateral dimension of the respective first and second therapeutic X-ray beam is not greater than a lateral dimension of the margin of the target 750 perpendicular to the respective first and second therapeutic X-ray beam (i.e., the collimator can be implemented to optimize the treatment X-ray beams to the size of the target 750 so as to minimize irradiation of healthy tissue surrounding the target 750). In other aspects, the step of collimating the first or second therapeutic X-ray beam comprises collimating the first or second therapeutic X-ray beam emitted by the X-ray source 100 in the power mode using a slit collimator 600 such that the first or second therapeutic X-ray beam is emitted onto the target 750 as a linear area. In yet other aspects, the step of collimating the first or second imaging X-ray beam emitted by the X-ray source 100 in the imaging mode using a cone beam collimator 625 such that the first or second imaging X-ray beam is emitted onto the object as an elliptical area or a circular area. As disclosed herein, the steps of performing imaging and re-imaging the target and delivering the first and second X-ray dosages are accomplished using the same X-ray source 100.

In some instances, the step of actuating the X-ray source 100 in the power mode to emit a first or second X-ray dosage to the target 750, comprises actuating the X-ray source 100 in the power mode to emit a first or second X-ray dosage to the target 750 at a predetermined X-ray dosage rate for a predetermined time to provide a cumulative X-ray dosage to the target 750. In other instances, the method comprises tracking movement of the object during delivery of the first X-ray dosage; and adjusting the X-ray source 100 or the object to register the target 750 with the diagnostic image of the target 750, and to maintain the X-ray source 100 directed toward the target 750, in response to the tracked movement of the object (i.e., any movement of the object can be tracked via a fiducial marker attached to the object such that it remains in a known spatial relationship with the target 750 regardless of the movement of the object).

In order to perform such a method, aspects of the present disclosure comprise a corresponding multi-modal X-ray system 400 for providing X-ray therapy to a target 750 within an object, the target 750 being determined from and a diagnostic image of the target 750 being obtained by diagnostic imaging of the object. The X-ray system 400 comprises an X-ray detector 700; an X-ray source 100 including an anode 200 and a field emission cathode device 300 in spaced-apart relation with the anode 200 and arranged to emit electrons 305 toward the anode 200; and a controller 430 in communication with the X-ray source 100 and the X-ray detector 700. The controller 430 is configured to direct the X-ray source 100 to arrange the field emission cathode device 300 into an imaging mode to focus the electrons 305 on a first focal area on the anode 200, wherein the first focal area has a first focal area size; image the target 750 by actuating the X-ray source 100 in the imaging mode to emit an imaging X-ray beam such that the imaging X-ray beam interacts with the target 750 and is detected by the X-ray detector 700 to obtain a first X-ray image of the target 750; adjust the X-ray source 100 or the object to register the target 750 with the diagnostic image of the target 750, and direct the X-ray source 100 toward the target 750, in response to a comparison of the first X-ray image and the diagnostic image of the target 750; direct the X-ray source 100 to switch the arrangement of the field emission cathode device 300 into a power mode to focus the electrons 305 on a second focal area on the anode 200, wherein the second focal area has a second focal area size greater than the first focal area size; and actuate the X-ray source 100 in the power mode to emit a first therapeutic X-ray beam toward the target 750 to deliver a first X-ray dosage to the target 750.

In some aspects, the controller 430 is configured to direct the X-ray source 100 to switch the arrangement of the field emission cathode device 300 into the imaging mode, after the first X-ray dosage is delivered to the target 750; re-image the target 750 using the X-ray device to obtain a second X-ray image of the target 750; adjust the X-ray source 100 or the object to register the target 750 with the diagnostic image of the target 750, and direct the X-ray source 100 toward the target 750, in response to a comparison of the second X-ray image and the diagnostic image of the target 750; direct the X-ray source 100 to switch the arrangement of the field emission cathode device 300 into a power mode to focus the electrons 305 on the second focal area on the anode 200; and actuate the X-ray source 100 in the power mode to emit a second therapeutic X-ray beam toward the target 750 to deliver a second X-ray dosage to the target 750.

In some aspects, the X-ray detector 700 is arranged to oppose the X-ray source 100 when the field emission cathode device 300 of the X-ray source 100 is in a transmission imaging mode, while in other aspects, the X-ray detector 100 is arranged adjacent to the X-ray source 100 when the field emission cathode device 300 of the X-ray source 100 is in a backscatter imaging mode. In some aspects, the X-ray detector 700 is arranged to not receive the first or second therapeutic X-ray beam when the field emission cathode device 300 of the X-ray source 100 is in the power mode (e.g., the X-ray detector 700 is deactuated or otherwise moved out of the effective path of the X-ray beam(s) emitted by the X-ray source 100.

In some aspects, the controller 430 is configured to adjust the X-ray source 100 or the object by adjusting a distance between the X-ray source 100 and the object, adjusting an angular position of the X-ray source 100 in an orbit about the object, or adjusting a position of the X-ray source 100 or the object relative to the other, laterally and non-parallel to the imaging X-ray beam.

In some aspects, a margin of the target 750 is determined from the diagnostic imaging, and the system 400 comprises a collimator arranged between the X-ray source 100 and the object, wherein the collimator is configured to collimate the first or second therapeutic X-ray beam emitted by the X-ray source 100 in the power mode such that a lateral dimension of the respective first and second therapeutic X-ray beam is not greater than a lateral dimension of the margin of the target 750 perpendicular to the respective first and second therapeutic X-ray beam. Such a collimator can comprise a slit beam collimator 600 configured to collimate the first or second therapeutic X-ray beam emitted by the X-ray source 100 in the power mode such that the first or second therapeutic X-ray beam is emitted onto the target 750 as a linear area. In other aspects, such a collimator can comprise a cone beam collimator 625 arranged between the X-ray source 100 and the object, and configured to collimate the first or second imaging X-ray beam emitted by the X-ray source 100 in the imaging mode such that the first or second imaging X-ray beam is emitted onto the object as an elliptical area or a circular area. In some aspects, imaging and re-imaging the target 750 and delivering the first and second X-ray dosages is performed using the same X-ray source 100.

In some aspects, the controller 430 is configured to actuate the X-ray source 100 in the power mode to emit a first or second X-ray dosage to the target 750 at a predetermined X-ray dosage rate for a predetermined time to provide a cumulative X-ray dosage to the target 750. In other aspects, a tracking device (e.g., a fiducial marker detectable by the X-ray device in the imaging mode, or a fiducial marker and detector system in communication with the controller 430) is included in communication with the controller 430 and is configured to track movement of the object during delivery of the first X-ray dosage, wherein the controller 430 is configured to adjust the X-ray source 100 or the object to register the target 750 with the diagnostic image of the target 750, and to maintain the X-ray source 100 directed toward the target 750, in response to the tracked movement of the object.

Many modifications and other embodiments of the disclosures set forth herein will come to mind to one skilled in the art to which these disclosures pertain having the benefit of the teachings presented in the foregoing descriptions and the associated drawings. For example, instead of a single X-ray source 100/single X-ray detector 700 pair mounted to the gantry 410, some aspects of the disclosure may include multiple X-ray sources 100 and corresponding X-ray detectors 700 mounted on the gantry 410, as shown in FIG. 10. Such an arrangement could provide, for example, multiple radiation therapy treatment to the target/tumor 750 from different directions/angles to achieve a higher dose rate over a shorter treatment time. Imaging of the target 750 could also be performed from different directions/angles and/or the target 750 could be imaged by one X-ray source 100/X-ray detector 700 pair while (e.g., simultaneously with) a second X-ray source 100 is providing radiation therapy treatment to the target/tumor 750.

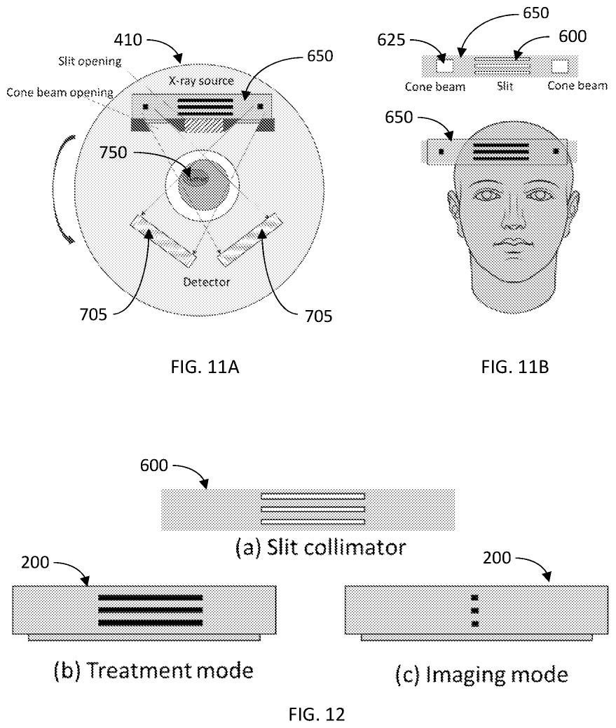

In another example, an X-ray source 100 can be configured to generate both the treatment and the imaging X-ray beams at the same time, as shown in FIGS. 11A and 11B. More particularly, in one example, the X-ray source 100 may be configured/programmed for certain field emission cathodes 310 of the field emission cathode device 300 to form two small focal spots on the anode 200 for imaging purposes, with those two small focal spots being on opposing sides of three large focal spots/lines formed by actuating other groups of the field emission cathodes for radiation therapy treatment purposes. Such a configuration may allow the radiation therapy treatment X-ray beams and imaging X-ray beams to be independently collimated with a single, stationary collimator assembly 650 (e.g., including cone beam collimators 625 and slit collimators 600 in a single assembly, and arranged to minimize the overlap between imaging X-ray beams and treatment X-ray beams). The imaging X-ray beams can be actuated during the treatment (e.g., while the radiation therapy treatment X-ray beam(s) are actuated) to provide real time imaging guidance and feedback, without interrupting the radiation treatment. Two corresponding X-ray detectors 705 can also be mounted to the gantry 410 and used to capture the imaging X-ray beams to provide the imaging guidance and feedback during the treatment.

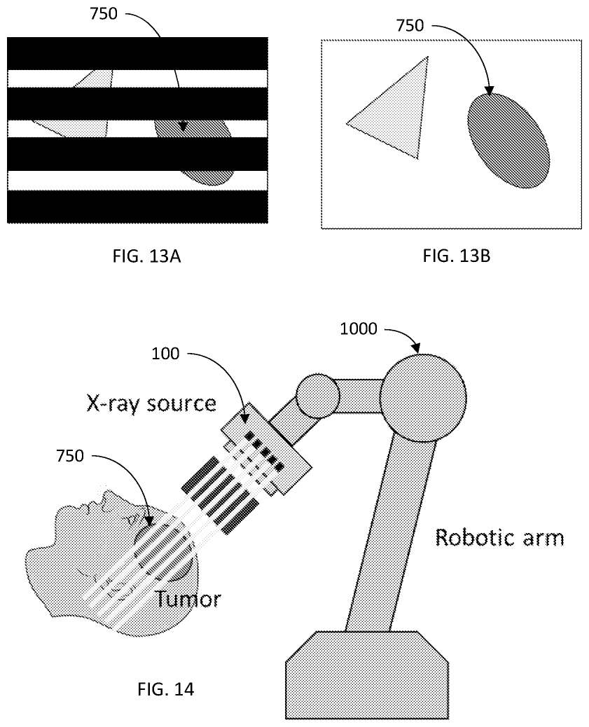

In still another example, a slit collimator 600 could also be implemented for imaging purposes during the radiation therapy treatment. As shown in FIG. 12(a), a single slit collimator 600, which may have one or more slits) is used for both treatment and imaging. During the radiation treatment procedure, the X-ray source 100 can be configured and arranged such that groups of field emission cathodes 310 are actuated to form large focal spots/lines, wherein the large focal spots/lines are aligned with the slits defined by the collimator 600 for treatment X-ray beam generation, as shown in FIG. 12(b). Between sub-treatments of the target/tumor 750, the field emission cathode device 300 can be switched to the imaging mode such that one or a small group of field emission cathodes 310 are actuated to form small focal spots on the anode 200. As shown in FIG. 12(c), in imaging mode, the small focal spots are conducive to high resolution imaging capture. The pattern of the small focal spots remains selected to match the pattern of the slits in the slit collimator 600. In such an instance, the imaging X-ray beams directed through the slits of the slit collimator 600 and detected by the X-ray detector 700 will result in the generation of an incomplete/partial image of the target 750, as shown in FIG. 13A, due to the interference of the portions of the slit collimator 600 defining the slits (e.g., the bars of the slit collimator). The partial image, including portions of the target/tumor 750 can still be registered to the original diagnostic image of the target/tumor 750 (see, e.g., FIG. 13B), and can therefore still provide imaging guidance and feedback during the radiation therapy treatment.

In yet another example, as shown in FIG. 14, the X-ray source 100 may be mounted on a robotic arm 1000, instead of a gantry, to allow for greater flexibility and freedom of movement in positioning the X-ray source 100 in three-dimensional (3D) space for optimal interaction with the target/tumor 750. In such a configuration, a corresponding X-ray detector may be mounted to another robotic arm (not shown) to provide imaging guidance and feedback during the radiation treatment and/or while the X-ray source 100 is in motion via movement of the robotic arm 1000. In other instances, a corresponding X-ray detector can also be mounted on the same robotic arm 1000 adjacent to the X-ray source 100, wherein the X-ray source 100 may be configured to utilize a backscatter mode for X-ray imaging, imaging guidance, and feedback purposes.

Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method of providing multi-modal X-ray therapy to a target within an object using an X-device including an X-ray source and an X-ray detector, the X-ray source including an anode and a field emission cathode device in spaced-apart relation with the anode and arranged to emit electrons toward the anode, and the target being determined from and a diagnostic image of the target being obtained by diagnostic imaging of the object, the method comprising:
arranging the field emission cathode device of the X-ray source into an imaging mode to focus the electrons on a first focal area on the anode, the first focal area having a first focal area size;
imaging the target by actuating the X-ray source in the imaging mode to emit an imaging X-ray beam toward the X-ray detector such that the imaging X-ray beam interacts with the target to obtain a first X-ray image of the target;
adjusting the X-ray source or the object to register the target with the diagnostic image of the target, and to direct the X-ray source toward the target, in response to a comparison of the first X-ray image and the diagnostic image of the target;
switching the arrangement of the field emission cathode device into a power mode to focus the electrons on a second focal area on the anode, the second focal area having a second focal area size greater than the first focal area size; and
actuating the X-ray source in the power mode to emit a first therapeutic X-ray beam toward the target to deliver a first X-ray dosage to the target.

2. The method of claim 1, comprising:
switching the arrangement of the field emission cathode device of the X-ray source into the imaging mode, after delivery of the first X-ray dosage to the target;
re-imaging the target using the X-ray device to obtain a second X-ray image of the target;
adjusting the X-ray source or the object to register the target with the diagnostic image of the target, and to direct the X-ray source toward the target, in response to a comparison of the second X-ray image and the diagnostic image of the target;
switching the arrangement of the field emission cathode device into the power mode to focus the electrons on the second focal area on the anode; and
actuating the X-ray source in the power mode to emit a second therapeutic X-ray beam toward the target to deliver a second X-ray dosage to the target.

3. The method of claim 1, comprising arranging the X-ray detector to oppose the X-ray source when the field emission cathode device of the X-ray source is in a transmission imaging mode.

4. The method of claim 1, comprising arranging the X-ray detector adjacent to the X-ray source when the field emission cathode device of the X-ray source is in a backscatter imaging mode.

5. The method of claim 2, comprising arranging the X-ray detector to not receive the first or second therapeutic X-ray beam when the field emission cathode device of the X-ray source is in the power mode.

6. The method of claim 2, wherein adjusting the X-ray source or the object comprises adjusting a distance between the X-ray source and the object, adjusting an angular position of the X-ray source in an orbit about the object, or adjusting a position of the X-ray source or the object relative to the other, laterally and non-parallel to the imaging X-ray beam.

7. The method of claim 2, wherein a margin of the target is determined from the diagnostic imaging, and wherein the method comprises collimating the first or second therapeutic X-ray beam emitted by the X-ray source in the power mode such that a lateral dimension of the respective first and second therapeutic X-ray beam is not greater than a lateral dimension of the margin of the target perpendicular to the respective first and second therapeutic X-ray beam.

8. The method of claim 7, wherein collimating the first or second therapeutic X-ray beam comprises collimating the first or second therapeutic X-ray beam emitted by the X-ray source in the power mode using a slit collimator such that the first or second therapeutic X-ray beam is emitted onto the target as a linear area.

9. The method of claim 2, wherein actuating the X-ray source in the power mode to emit a first or second X-ray dosage to the target, comprises actuating the X-ray source in the power mode to emit a first or second X-ray dosage to the target at a predetermined X-ray dosage rate for a predetermined time to provide a cumulative X-ray dosage to the target.

10. The method of claim 2, comprising performing imaging and re-imaging the target and delivering the first and second X-ray dosages using the same X-ray source.

11. The method of claim 2, comprising collimating the first or second imaging X-ray beam emitted by the X-ray source in the imaging mode using a cone beam collimator such that the first or second imaging X-ray beam is emitted onto the object as an elliptical area or a circular area.

12. The method of claim 1, comprising:
tracking movement of the object during delivery of the first X-ray dosage; and
adjusting the X-ray source or the object to register the target with the diagnostic image of the target, and to maintain the X-ray source directed toward the target, in response to the tracked movement of the object.

13. A multi-modal X-ray system for providing X-ray therapy to a target within an object, the target being determined from and a diagnostic image of the target being obtained by diagnostic imaging of the object, the X-ray system comprising:
an X-ray detector;
an X-ray source including an anode and a field emission cathode device in spaced-apart relation with the anode and arranged to emit electrons toward the anode; and
a controller in communication with the X-ray source and the X-ray detector, the controller being configured to:
direct the X-ray source to arrange the field emission cathode device into an imaging mode to focus the electrons on a first focal area on the anode, the first focal area having a first focal area size;
image the target by actuating the X-ray source in the imaging mode to emit an imaging X-ray beam such that the imaging X-ray beam interacts with the target and is detected by the X-ray detector to obtain a first X-ray image of the target;
adjust the X-ray source or the object to register the target with the diagnostic image of the target, and direct the X-ray source toward the target, in response to a comparison of the first X-ray image and the diagnostic image of the target;
direct the X-ray source to switch the arrangement of the field emission cathode device into a power mode to focus the electrons on a second focal area on the anode, the second focal area having a second focal area size greater than the first focal area size; and
actuate the X-ray source in the power mode to emit a first therapeutic X-ray beam toward the target to deliver a first X-ray dosage to the target.

14. The system of claim 13, wherein the controller is configured to:
direct the X-ray source to switch the arrangement of the field emission cathode device into the imaging mode, after the first X-ray dosage is delivered to the target;
re-image the target using the X-ray device to obtain a second X-ray image of the target;
adjust the X-ray source or the object to register the target with the diagnostic image of the target, and direct the X-ray source toward the target, in response to a comparison of the second X-ray image and the diagnostic image of the target;
direct the X-ray source to switch the arrangement of the field emission cathode device into a power mode to focus the electrons on the second focal area on the anode; and
actuate the X-ray source in the power mode to emit a second therapeutic X-ray beam toward the target to deliver a second X-ray dosage to the target.

15. The system of claim 13, wherein the X-ray detector is arranged to oppose the X-ray source when the field emission cathode device of the X-ray source is in a transmission imaging mode.

16. The system of claim 13, wherein the X-ray detector is arranged adjacent to the X-ray source when the field emission cathode device of the X-ray source is in a backscatter imaging mode.

17. The system of claim 14, wherein the X-ray detector is arranged to not receive the first or second therapeutic X-ray beam when the field emission cathode device of the X-ray source is in the power mode.

18. The system of claim 14, wherein the controller is configured to adjust the X-ray source or the object by adjusting a distance between the X-ray source and the object, adjusting an angular position of the X-ray source in an orbit about the object, or adjusting a position of the X-ray source or the object relative to the other, laterally and non-parallel to the imaging X-ray beam.

19. The system of claim 14, wherein a margin of the target is determined from the diagnostic imaging, and wherein the system comprises a collimator arranged between the X-ray source and the object, the collimator being configured to collimate the first or second therapeutic X-ray beam emitted by the X-ray source in the power mode such that a lateral dimension of the respective first and second therapeutic X-ray beam is not greater than a lateral dimension of the margin of the target perpendicular to the respective first and second therapeutic X-ray beam.

20. The system of claim 19, wherein the collimator comprises a slit beam collimator configured to collimate the first or second therapeutic X-ray beam emitted by the X-ray source in the power mode such that the first or second therapeutic X-ray beam is emitted onto the target as a linear area.

21. The system of claim 14, wherein the controller is configured to actuate the X-ray source in the power mode to emit a first or second X-ray dosage to the target at a predetermined X-ray dosage rate for a predetermined time to provide a cumulative X-ray dosage to the target.

22. The system of claim 14, wherein imaging and re-imaging the target and delivering the first and second X-ray dosages is performed using the same X-ray source.

23. The system of claim 14, comprising a cone beam collimator arranged between the X-ray source and the object, and configured to collimate the first or second imaging X-ray beam emitted by the X-ray source in the imaging mode such that the first or second imaging X-ray beam is emitted onto the object as an elliptical area or a circular area.

24. The system of claim 13, comprising a tracking device in communication with the controller and configured to track movement of the object during delivery of the first X-ray dosage, wherein the controller is configured to adjust the X-ray source or the object to register the target with the diagnostic image of the target, and to maintain the X-ray source directed toward the target, in response to the tracked movement of the object.

25. A method of providing multiple modes of an X-ray source device, the X-ray source device including an anode and a field emission cathode device in spaced-apart relation with the anode and arranged to emit electrons toward the anode, comprising:
arranging the field emission cathode device into an imaging mode to focus the electrons on a first focal area on the anode, the first focal area having a first focal area size; and
reversibly switching the arrangement of the field emission cathode device into a power mode to focus the electrons on a second focal area on the anode, the second focal area having a second focal area size greater than the first focal area size.

26. The method of claim 25, wherein the field emission cathode device comprises a plurality of individually-controllable field emission cathodes, and wherein arranging the field emission cathode device into the imaging mode comprises actuating a first amount of the field emission cathodes to direct the electrons emitted thereby to the first focal area on the anode.

27. The method of claim 26, wherein the field emission cathode device comprises a plurality of individually-controllable field emission cathodes, and wherein switching the arrangement of the field emission cathode device into the power mode comprises actuating a second amount of the field emission cathodes, the second amount being greater than the first amount, to direct the electrons emitted thereby to the second focal area.

28. The method of claim 27, wherein directing the electrons to the second focal area comprises directing the electrons from each of the second amount of field emission cathodes to respective corresponding focal areas on the anode, the respective corresponding focal areas being adjacently disposed and arranged to form the second focal area on the anode.

29. The method of claim 28, wherein each of the second amount of the field emission cathodes emits an electron current directed to the anode upon actuation thereof, and wherein the method comprises modulating the current emitted by a selected one or more of the second amount of field emission cathodes so as to modulate an intensity of the electrons emitted thereby to the second focal area.

30. A multi-modal X-ray source device, comprising:
an anode; and
a field emission cathode device in spaced-apart relation with the anode and arranged to emit electrons toward the anode, the field emission cathode device being switchable between an imaging mode having the electrons focused on a first focal area on the anode, the first focal area having a first focal area size, and a power mode having the electrons focused on a second focal area on the anode, the second focal area having a second focal area size greater than the first focal area size.

31. The device of claim 30, wherein the field emission cathode device comprises a plurality of individually-controllable field emission cathodes configured such that the imaging mode comprises a first amount of the field emission cathodes actuated to direct the electrons emitted thereby to the first focal area on the anode.

32. The device of claim 31, wherein the field emission cathode device is configured such that the power mode comprises a second amount of the field emission cathodes, the second amount being greater than the first amount, actuated to direct the electrons emitted thereby to the second focal area.

33. The device of claim 32, wherein the second amount of field emission cathodes is arranged to direct the electrons emitted thereby to respective corresponding focal areas on the anode, the respective corresponding focal areas being adjacently disposed and arranged to form the second focal area on the anode.

34. The device of claim 33, wherein each of the second amount of the field emission cathodes emits an electron current directed to the anode upon actuation thereof, and wherein the electron current emitted by a selected one or more of the second amount of field emission cathodes is modulatable so as to modulate an intensity of the electrons emitted thereby to the second focal area.
